(19) United States
(12) Plant Patent Application Publication
(10) Pub. No.: US 2002/0129415 P1
(43) Pub. Date: Sep. 12, 2002

(54) Chrysanthemum named Resomee

(76) Inventor: Robert Noodelijk, Woubrugge (NL)

Correspondence Address: PARKHURST & WENDEL, 1421 PRINCE STREET, SUITE 210, ALEXANDRIA, VA, 22314, US

(21) Appl. No.: 09/757,640

(22) Filed: Jan. 11, 2001

(51) Int. Cl. A01H 5/00

(52) U.S. Cl. PLT/291

(57) ABSTRACT

A Chrysanthemum plant named Resomee characterized by its large sized blooms with pink ray-florets.

### RELATED APPLICATIONS

The present application is related to the following co-pending applications: Application no. Filing Date Title

### BACKGROUND OF THE INVENTION

The new plant of the present invention comprises a new and distinct cultivar of Chrysanthemum plant that is a cross of unknown Dendranthema Grandiflora parents. The new and distinct cultivar was a seedling resulting from an open pollination among groups of chrysanthemum cultivars maintained under the control of the inventor for breeding purposes. The new and distinct cultivar was discovered and selected as one flowering plant by Rob Noodelijk in a controlled environment (greenhouse) in Rijsenhout Holland in September 1998. The first act of asexual reproduction of Resomee was accomplished when vegetative cuttings were taken from the initial selection in November 1998 in a controlled environment in Rijsenhout Holland.

### SUMMARY OF THE INVENTION

The present invention is a new and distinct variety of chrysanthemum bearing large sized blooms with pink ray-florets.

### BRIEF DESCRIPTION OF THE DRAWINGS

The present invention of a new and distinct variety of chrysanthemum is shown in the accompanying drawings, the color being as nearly true as possible with color photographs of this type.

FIG. 1 shows a stem of the cultivar in full bloom.

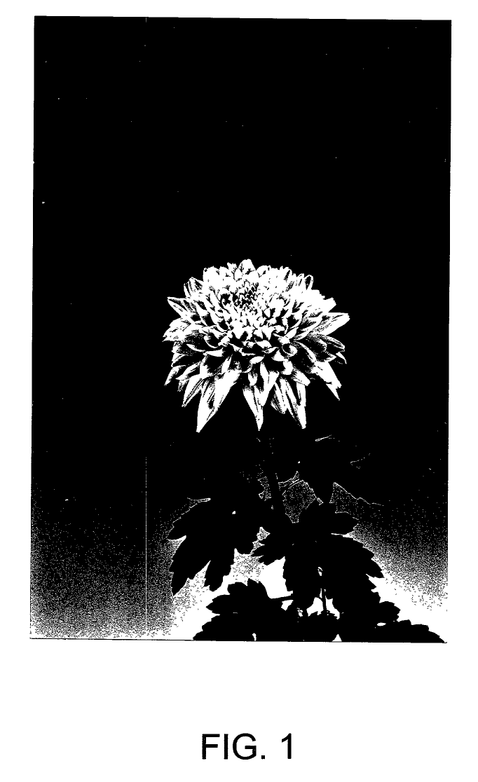

FIG. 2 shows 2 stages of the bloom.

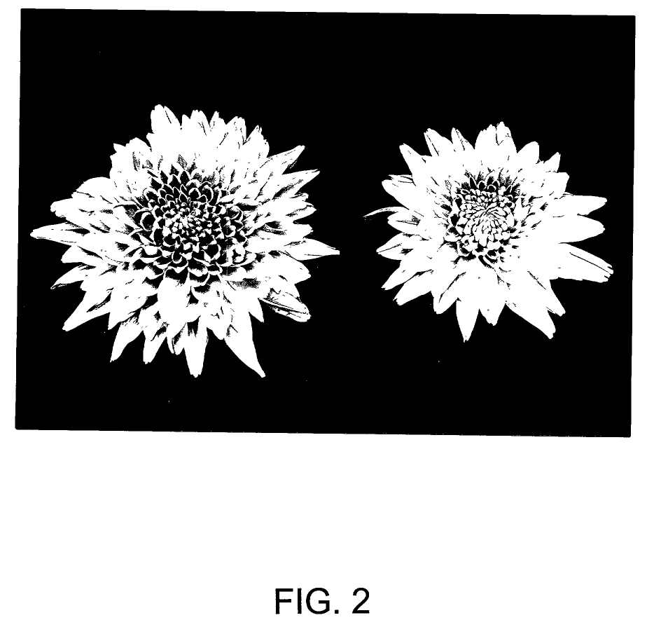

FIG. 3 shows the foliage of the cultivar.

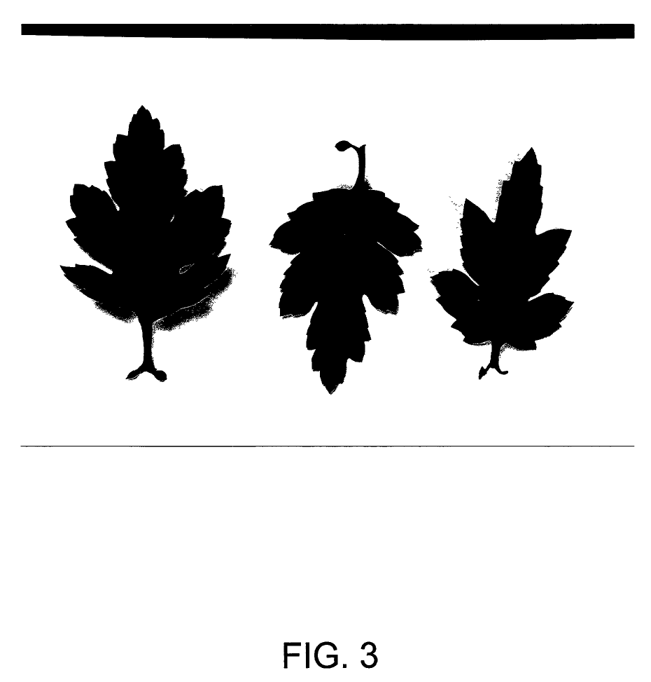

### DESCRIPTION OF THE INVENTION

This new variety of chrysanthemum is of the botanical classification Dendranthema Grandiflora. The observations and measurements were gathered from plants grown in a greenhouse in Rijsenhout Holland in a photo-periodic controlled crop. The photo-periodic response time in this crop was 56 days. The variety has been tested as a disbudded variety only. The description refers to this. This new variety produces large sized blooms with pink ray-florets blooming on the plant for 21 days. This new variety of chrysanthemum has been found to retain its distinctive characteristics throughout successive propagations however the phenotype may vary significantly with variations in environment such as light intensity and temperature. To show the phenotype as described Resomee may be planted without assimilation lightning between week 50 and 40 under greenhouse conditions in Holland. With assimilation lightning (minimum level 2500 lux) it may be planted year round under greenhouse conditions in Holland.

The following is a description of the plant and characteristics that distinguish Resomee as a new and distinct variety.

The color designations are taken from the plant itself. Accordingly, any discrepancies between the color designations and the colors depicted in the photographs are due to photographic tolerances.

Botanical Description of CULTIVAR Resomee

| | |
|---|---|
| Bud | |
| Size | Not applicable |
| Outside Color | Not applicable |
| Bloom | |
| Size | Large |
| Fully Expanded | 11 cm |
| borne | |
| number of blooms per single stem | Not applicable |
| Peduncle length | Not applicable |
| Peduncle lateral shoot attachment | Not applicable |
| Peduncle lateral shoot angle | Not applicable |
| Form | Decorative |
| Performance (blooming period) | 21 days |
| Color | |
| Center of the flower (ray-florets) | Immature purple 75A mature |
| | purple 75B |
| Color of upper surface of the majo- | Purple 75B |
| rity of the ray-florets | |
| Color of the lower surface of the | Purple 76D |
| majority of the ray-florets | |
| Tonality from Distance | A large pink decorative bloom |
| Discoloration | None |
| Ray florets | |
| Texture | Upper and under side smooth |
| Number | 180-190 |
| Cross-section | Flat to concave |
| Longitudinal axis of majority | Straight |
| Shape of tip | Pointed |
| Fragrance | Typical chrysanthemum |
| Disc Diameter (when visible) | |
| Reproductive Organs | |
| Stamen (present in disc florets only) | Not present |
| Pollen | Not present |
| Styles (present in both ray and disc | Yellow-green, thin |
| florets) | |
| Style Length | +/− 3 mm |
| Stigmas | Yellow-green |
| Stigma Width | +/− 1 mm |
| Ovaries | Enclosed in calyx |
| Plant | |
| Form | Spray mum meant for erect |
| | culture. Herbaceous |
| Growth | Vigorous |
| Height | 125 cm |
| Stem Color | Green 138A |
| Stem Strength | Strong |
| Stem Brittleness | Absent |
| Stem Anthocyanin Coloration | Absent |
| Intemodes | 2,5-3 cm |
| Flowering Response(photo-periodic | 56 days |
| controlled crop, no natural growing) | |
| Foliage | |
| Color | Upper side green 139 A |
| | under side green 137 A |
| Size | large; length +/− 9,5 cm, |
| | width +/ 8 cm |
| Quantity (number per stem) | 28-30 |
| Shape | Round |
| Texture | Fleshy |
| Ribs and Veins | Ribs and veins well developed |
| Edge | Crenated |
| Shape of Base of Sinus Between | Acute |
| Lateral Lobes | |
| Margin of Sinus Between Lateral | Diverging |
| Lobes | |
| Shape of Base | Truncate |
| Apex | Cuspidate |

I claim:

1. A new and distinct variety of chrysanthemum plant as described and illustrated.
